(19) United States
(12) Patent Application Publication
(10) Pub. No.: US 2007/0239255 A1
(43) Pub. Date: Oct. 11, 2007

(54) System and device for helical stent delivery

(76) Inventor: Richard Allen Hines, Stilwell, KS (US)

Correspondence Address: RICHARD A. HINES, 16525 ORCHARD LANE, STILWELL, KS, 66085, US

(21) Appl. No.: 11/784,228

(22) Filed: Apr. 6, 2007

Related U.S. Application Data
(60) Provisional application No. 60/790,423, filed on Apr. 7, 2006.

(51) Int. Cl. A61F 2/84; A61F 2/88

(52) U.S. Cl. 623/1.12; 623/1.22

(57) ABSTRACT

A self expanding helical medical device is delivered intralumenaly through a very flexible small diameter catheter. The delivery is made possible by the use of a novel locking wrapper that maintains the stent on the guidewire at the delivery diameter and then, in a controlled and gradual fashion, automatically releases the stent to expand to the artery diameter as the stent/wrapper is moved out of the catheter. The delivery system is particularly useful for delivering a stent with a large solid surface area to the neck of a neurovascular aneurysm to cure the aneurysm.

### CROSS-REFERENCE TO RELATED APPLICATIONS

This application claims priority based upon provisional application 60/790,423 filed Apr. 7, 2006.

### TECHNICAL FIELD

The present invention is directed to the field of medical and veterinary endovascular delivery of stents, and more particularly, to delivery of helical self-expanding stents. The method is well suited for the delivery of stents for the treatment of neurovascular aneurysms.

### BACKGROUND OF THE INVENTION

Neurovascular aneurysms are currently treated by two methods. The original treatment is an open surgical procedure called clippings that removes the aneurysm from the circulatory system by placing a clip at the base of the aneurysm. A newer, less invasive, endovascular procedure called coiling packs the aneurysm with flexible platinum coils that reduce blood circulation in the aneurysm and, thereby, trigger a thrombus in the aneurysm that may stop blood leakage and reduce the threat of the aneurysm bursting. Self-expanding Nitinol stents are sometimes used with coiling. The stents form a lattice over the neck of the aneurysm to help keep coils from prolapsing into the parent artery.

An aneurysm is formed when a weak spot in an artery stretches so thin that it is in danger of bursting from the pressure of the blood it contains. It forms a bulge or a ballooning area that may leak or rupture. An aneurysm that ruptures in a brain artery causes a stroke. Aneurysms that have wide openings at their base are called “wide neck” aneurisms and are the most difficult to treat. Wide neck aneurysms generally are defined as having a neck ≧4 mm or a dome-to-neck ratio <2.

About 5 million people in the United States currently have a brain aneurysm, and about 25 percent of these are “wide neck” aneurysms. In the United States it is estimated that as many as 18 million people will develop a brain aneurysm during their lifetime. It is estimated that every year more than 30,000 people suffer from ruptured brain aneurysms. Ten to 15 percent of these patients die before reaching the hospital. More than 50 percent die within the first 30 days after rupture. Of those who survive, approximately half suffer some permanent neurological deficit.

An aneurysm may cause pain or other symptoms from pressure on surrounding tissue, but often aneurysms have no symptoms. Aneurysms may be discovered during routine medical exams or diagnostic procedures for other health problems, but most often people are unaware of a problem until a rupture occurs. As relatively simple, viable treatments for aneurysms are developed physicians will look for and find more silent aneurysms and treat them before they cause problems.

The potential benefits of aneurysm treatments by clipping or coiling often do not outweigh the risks, especially for patients in whom remaining life expectancy is less than 20 years.

Neurosurgical clipping involves a craniotomy, an invasive, open surgical procedure with high risk. During this procedure, the arteries are exposed and one or more clips are applied across the neck of the aneurysm to stop blood from flowing into the aneurysm. The risk of a craniotomy is exacerbated in patients with a recent brain injury as well as in elderly or medically complicated patients. There is potential for further injury to the brain and additional neurological defect.

Endovascular coiling is a less invasive, non-surgical technique that involves inserting detachable platinum coils via a catheter into the aneurysm. The goal of endovascular coiling is to tightly pack coils inside the aneurysm to restrict blood flow within the aneurysm, and thus form a thrombus. The formation of a thrombus leaves little or no liquid in the aneurysm, eliminating the potential for the aneurysm to expand, leak or burst. The use of platinum allows the coils to be visible via X-ray. Although the endovascular coiling process plays a role in the treatment of brain aneurysms, the process has limitations. When platinum coils fill the aneurysm, the aneurysm size will remain basically the same and, therefore, it will continue to exert pressure on and interfere with surrounding tissue. This is known as the mass effect. The coiling procedure requires a long leaming process due to its technical difficulty. The process is effective in only a small percentage of aneurysms, such as the small neck aneurysms where the coils are more likely to stay in place. In other aneurysms, the coils are likely to protrude into the parent vessel with risk of clot formation and embolism.

Physicians have begun using stents or balloon-stent combinations in combination with coiling to improve the effectiveness of coiling. A balloon may sometimes be used to push the coils into or pack them into the aneurysm. With stent-assisted coiling, a stent is used to line the artery and form a screen to hold the platinum coils inside the aneurysm.

For direct treatment of neurovascular aneurysms, today's balloon-expandable or self-expanding stent designs are inadequate. Substantial open spaces in the walls of self-expanding stents and balloon-expandable stents do not sufficiently cover the aneurysm to block blood flow to the aneurysm. For example, in the stent-assisted coiling procedure, physicians currently use a thin self-expanding stent developed by the Boston Scientific Corporation. This product was approved for use by the FDA in 2002 for use with coils for the treatment of wide neck, intracranial, saccular aneurysms arising from a parent vessel with a diameter of ≧2 mm and ≦4.5 mm that are not amenable to treatment with surgical clipping. The flexibility of this Boston Scientific stent is derived from its very open design. It is intended to keep the coils in place, but the surface has a significant amount of open space and is not intended to block blood circulation across the neck of the aneurysm.

A stent with a greater percent solid area would restrict blood circulation into the aneurysm and trigger a thrombus in the aneurysm more effectively. In that event, the liquid aneurysm would solidify, eliminating the danger of rupture or leakage. If the aneurysm is filled with the thrombus only and no coils the aneurysm sac will shrink as the thrombus is absorbed, reducing pressure on the surrounding tissue.

Stents are generally designed as cylindrical shells comprised of interconnected elements or struts. The pattern of struts on the surface of the cylinder allows a stent to be crimped to a small diameter for delivery and to expand radially from the small delivery diameter to a larger placement diameter once positioned within the lumen. The final placement diameter of an expandable stent is generally between 2.5 and 4 times the delivery diameter. As a result, the surface of the expanded stent has a significant amount of open space. At the small delivery diameter, the metal struts of the stents cover about 50 percent of the surface area of the stent. At the expanded placement diameter, the area covered by the struts is only about 12 to 20 percent of the stent wall. Current research indicates that a dense stent will reduce flow into the aneurysm thus triggering a thrombus in the aneurysm. The open area of a typical stent, then, is a limitation with respect to treatment of an aneurysm.

Several additional types of stents and methods for making stents have been described previously. For example, the documents U.S. Pat. Nos. 6,527,919, 6,080,191, 6,007,573, and 6,669,719 discuss stents using methods involving rolled flat sheets. U.S. Pat. No. 6,689,159 discusses a radially expandable stent with cylindrical elements and where expansion occurs when the stress of compression is removed. Stents manufactured from a flat sheet tend to have a high percent solid area but have limited longitudinal flexibility and since their storage volume for delivery is no longer than the stent they tend to have a large delivery diameter that limits the aneurysm they could reach.

U.S. Pat. Nos. 6,361,558 and 6,063,111, included herein by reference, discuss a helical stent that expands into a relaxed helical shape when released from a catheter. This stent has the potential for a very flexible delivery configuration through a small diameter catheter but the method for pushing the stent from the catheter will not work. The stretched helical stent will have a tendency to shorten and expand in diameter. This tendency will lock the stent into the catheter tube. The deployment method described in the '558 and the '111 patents use a “pusher” to push the stent from the catheter. Pushing on the proximal end of the stent will only increase the tendency for the stent to expand in diameter and lock into the catheter tube preventing the stent from being deployed. Due to these barriers, helical, self expanding stents have not found commercial success.

Additional methods which artificially solidify aneurysms have been suggested. For example, the document U.S. Pat. No. 6,569,190 discusses a method for treating aneurysms that involves filling an aneurysmal sac with a non-particulate agent or fluid that solidifies in situ. This process leaves a permanent lump cast in the volume of the aneurysm. The lump is an undesirable side effect of solidification of the aneurysm volume. Additionally, the filling agent has a tendency to leak or partially break off into the parent artery, thereby creating a risk of blockage.

The pleated stent assembly of U.S. patent application Ser. No. 10/695,527 filed on Oct. 28, 2003 (the '527 Application) describes a stent for endovascular treatments that has advantages over other methods of treating aneurysms, in that, among other things, it provides a relatively solid area for closing off the aneurismal sac. The pleated stent of '527 being nearly solid over the full cylinder is limited in that it can not be used for aneurysm near side branch or perforator arteries that would also be occulted by the stent. The micro-pleated stent assembly of U.S. patent application Ser. No. 11/031899 filed on Jan. 7, 2005 (the '899 Application) describes a stent for endovascular treatments of aneurysms that may be patterned with a patch to block the neck of the aneurysm and avoid any near by perforators. The stents of both '527 and '899 are balloon-expandable and made from a ductile material. Being constructed from a ductile material limits their use to locations where they will not be crushed by external forces. The ductile material must also be thick enough to be strong enough to withstand vasospasms that could also deform the ductile stent that has no capability to spring back. The necessity to be thick and the use of a balloon for delivery limit the use of both '527 and '899 by limiting the minimum delivery diameter and by limiting longitudinal flexibility.

A need exists for non-surgical aneurysm treatments with improved deliverability and effectiveness. The risk associated with open surgery often outweighs the potential benefits, particularly if coiling is feasible. Coiling is limited to narrow neck aneurysm and is a technically challenging procedure requiring poking a guide wire and many, often over 20 coils into the sac of a fragile aneurysm. Coils can prolapse into the parent artery causing a life-threatening thrombus to form. Stents of the '527 and '899 applications are limited to intracranial applications and have limited deliverability. Prior art delivery methods for helical stents such as those described in U.S. Pat. Nos. 6,361,558 and 6,063,111 are not effective. This lack of acceptable delivery method has prevented commercial development of potentially valuable helical stent treatments for aneurysms. Therefore, the existing technology for aneurysm treatment, and specifically for the delivery of helical stents to treat aneurysms, carries a number of limitations.

### BRIEF SUMMARY OF THE INVENTION

The current invention consists of a method and related hardware for the delivery, i.e., discharge, of a helical stent stored in a stretched form in a delivery catheter. In the preferred embodiment, the stent is delivered intravenously to a neurovascular aneurysm and placed so that the stent covers the neck of the aneurysm so as to initiate a thrombus in the aneurysm to start a healing process. The delivery catheter would include a guidewire with a conventional radiopaque flexible tip to aid in positioning the catheter. The stent in the catheter is stretched over the guidewire many times its relaxed length to reduce its diameter and improve flexibility for delivery. The ends of the stent essentially do not rotate relative to each other as the stent is stretched or delivered. The elastic limit of the stent material is not exceeded so that the stent will expand to the artery diameter when expelled from the catheter tube.

When tension on the stretched stent is released, the stent will expand in diameter to its natural diameter unless restrained. If restrained directly in the catheter tube, the tendency to expand will lock the stent into the catheter tube preventing its delivery. The current invention uses a thin plastic cylindrical wrapper to restrain the stent over the guidewire at a size that will slide through the catheter tube. A locking mechanism holds the wrapper in a cylindrical form while the wrapper and stent are in the catheter. The locking mechanism automatically unlocks as the stent is moved beyond the catheter tube allowing the stent to expand to meet the artery. The expanded stent provides a sufficiently solid covering over the neck of the aneurysm to trigger a thrombus in the aneurysm and start a healing process that stops blood leakage and reduces the threat of a rupture.

### DESCRIPTION OF THE DRAWINGS

FIG. 1A shows a partial cross-section of the distal tip of the delivery catheter with a partially deployed stent.

FIG. 1B shows a partial cross-section of a more proximal area of the delivery catheter of FIG. 1A.

FIG. 2 shows a transverse cross-section through the delivery catheter.

FIG. 3 shows the stent and catheter from FIG. 1A and FIG. 1B located in an artery with the stent partially delivered at the site of an aneurysm.

### DETAILED DESCRIPTION OF PREFERRED EMBODIMENT

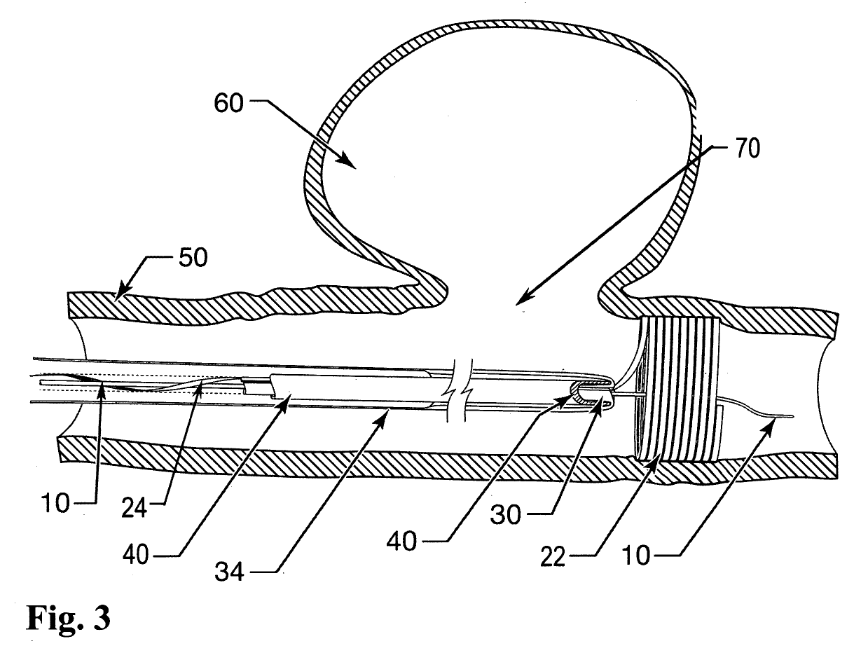

The current invention consists of a method and related hardware for the delivery, i.e., discharge, of a helical stent stored in a stretched form in a delivery catheter. In the preferred embodiment the stent, shown in FIG. 3 as an expanded helical coil 22 and as a stretched helical coil 24, is delivered intravenously to a neurovascular aneurysm 60 on artery 50. The stent is positioned so that the expanded stent 22 covers the neck 70 of the aneurysm 60 to initiate a thrombus in the aneurysm to start a healing process. The delivery catheter would include a guidewire 10 with a conventional radiopaque flexible tip to aid in positioning the catheter. In the catheter tube 40, the stent is stretched over guidewire 10 so that the stretched stent 24 is many times the stent's relaxed length to reduce its diameter and improve flexibility for delivery. The ends of the stent essentially do not rotate relative to each other as the stent is stretched or delivered. The elastic limit of the stent material is not exceeded so that the stent will have a tendency to self expand to the artery diameter when expelled from the catheter tube 40.

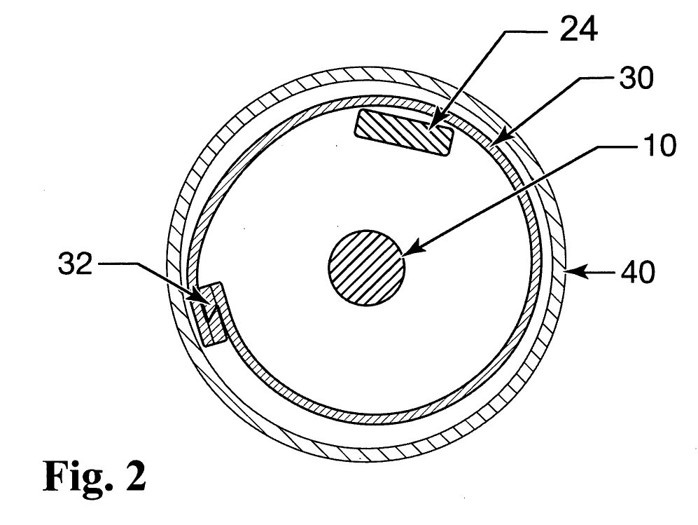

When tension on the stretched stent 24 is released, the stent will expand in diameter to its natural diameter unless restrained. If restrained directly in the catheter tube 40 the tendency to expand will lock the stent into the catheter tube 40 preventing its delivery. This invention uses a thin plastic cylindrical wrapper 30 to restrain the stent 24 over the guidewire 10 at a size that will slide through the catheter tube 40. FIG. 2 shows the locking mechanism 32 that holds the wrapper 30 in a cylindrical form while the wrapper and stent are in the catheter. The locking mechanism 32 automatically unlocks as the stent is moved beyond the catheter tube 40 to allow the stent to expand to meet the artery 50. The expanded stent 22 provides a sufficiently solid covering over the neck of the aneurysm to trigger a thrombus in the aneurysm and start a healing process that stops blood leakage and reduces the threat of a rupture.

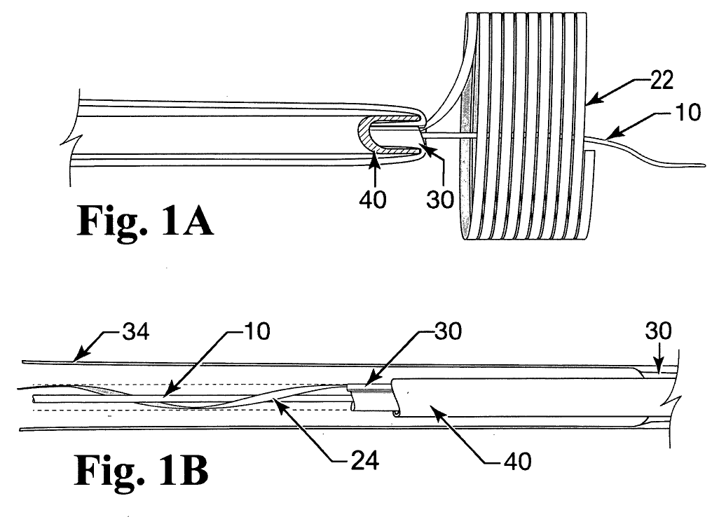

To assemble the delivery system the stent is placed on the guidewire 10 and stretched to the small delivery diameter. Before placement around the stent 24 the wrapper 30 is a thin elongated rectangular shape. The narrow dimension of the rectangle approximately equals the circumference of the stretched stent and the long dimension is somewhat longer than the length of the stretched stent 24. The wrapper 30 thus is closely wrapped around the stretched stent 24. Along both long edges of the wrapper are interlocking lock strips 32 which hold the wrapper in a cylindrical shape around the stretched stent 24. In FIG. 1A, FIG. 1B, and FIG. 3, the lock strip on one side faces the viewer and is shown with solid lines. The other lock strip is on the backside of the wrapper and is therefore shown with a dashed line. The lock strips 32, which form the wrapper locking mechanism, extend as two long strings 34 beyond the distal end of the wrapper. While maintaining tension on the stent to keep it in its stretched form, the catheter tube 40 is threaded over the guidewire 10, stretched stent 24, and wrapper 30. With the distal end of the catheter tube 40 aligned to the distal end of the stretched stent, the tension on the stent is released. As the stent shortens it expands in diameter until it is retained by the cylindrical wrapper 30. A snug fit between the catheter tube 40 and the wrapper 30 keeps the locking mechanism 32 engaged. The two strings 34 or extension of the locking strips extend out the distal end of the catheter tube 40. The strings fold back over the catheter tube 40 and extend just beyond the proximal end of the catheter.

Referring to FIG. 1A and 1B, the stent is moved out of the catheter 40 in small controlled steps. To deliver the stent to the aneurysm 60 the guidewire 10 is advanced just beyond the aneurysm in a standard manner. The catheter tube 40 is then advanced just beyond the aneurysm 60. To deploy the stent, the catheter tube 40 is pulled back while maintaining tension on the strings 34 and while moving the guidewire 10 in a distal direction. The combination of forces will expel the distal end of the stent and wrapper 30 from the catheter tube 40. When free of the catheter tube 40, the locking strips 32 will unlock and the stent will expand to the artery 50 diameter as shown in FIG. 3 as expanded stent 22. The relaxed size of the expanded stent 22, by design, will be slightly larger than the artery 50 to ensure that the expanded stent 22 seats properly against the artery wall. While holding the catheter tube 40 and wrapper strings 34 fixed, the guidewire 10 is pulled back (proximally) until its distal tip is near the distal end of the expanded stent 22. The process of pulling the catheter tube 40 and strings 34 back while dragging the stent 22 out with the distal movement of the guidewire 10 is repeated to release another length of the stent. The cycle is repeated until the stent 22 is completely free of the catheter 40 and wrapper 30.

When placing the stent, the wrapper 30 will necessarily be advanced in the catheter tube 40 more than the catheter tube 40 is moved proximally from the placed expanded stent 24, generally in the proportion the length of the stretched stent 22 is to the length of the expanded. stent 24. In other words, if, for example, the stent has a stretched length of 10 cm and an expanded length of 1 cm, the wrapper 30 generally will need to be advanced 10 cm for each 1 cm of expanded stent length, whereas the catheter generally will be pulled proximally 1 cm for each 1 cm of expanded length.

Although the natural distance between the coils of the expanded stent 22 is basically set by the elastic memory of the stent the actual spacing can be controlled by the individual placing the stent (the ‘interventionalist’) by controlling the position of the tip of the catheter as the coils are expelled from the catheter tube 40 and allowed to expand to the artery wall 50. With this control of the coil spacing, the interventionalist can expand the distance between the coils of the stent to span over perforator arteries that may be encountered. The deployment of the coils may be non-uniform to provide wider spacing at the anchor ends of the stent and tighter spacing at the neck of the aneurysm. Jailing, or blocking significant perforators could be avoided by judicially locating coils. The location of the nitinol coils would be made visible by radiopaque markers or a radiopaque central core within the nitinol. A radiopaque biodegradable coating on the stent could also be used.

When the stent is free of the catheter 40, the guidewire 10, catheter 40 and wrapper 30 are removed to complete the deployment of the stent. If, at any time before the last length of the stent is expelled from the catheter 40, it becomes necessary to abort the stent placement the entire catheter and stent may be removed by pulling the stent proximally back into the catheter tube 40.

The helical super elastic stent may be constructed from a round wire, a rectangular wire or it may be cut from a tube. In each case the percent solid area of the stent when expanded to the artery size will have a sufficiently large percent solid area to trigger a thrombus in the aneurysm. If the stent is formed from a tube, the tube may be thin film nitinol.

What is claimed and desired to be secured by Letters Patent is as follows:

1. A method of using a locking wrapper sleeve to hold a helical coil in a stretched form for intravenous delivery through a catheter to a site in a body vessel where the coil is released to elastically expand to the vessel diameter.

2. A vascular assembly for treating blood vessel aneurysms comprising:
a stent, a wrapper, and a catheter tube;
wherein said stent is a helical shape in a relaxed state, the stent defining a proximal opening and having a proximal end, the stent further having a body portion defining a lumen and a distal opening at a distal end defining a longitudinal distance from the proximal opening, the stent having a pitch defined as the longitudinal distance between adjacent loops in the helical state, wherein the stent can be stretched into a substantially linear, elongated helical shape, having a length between the proximal and the distal end and a circumference, and wherein said stent has a tendency to move from the substantially linear, elongated helical shape, to the relaxed helical shape; and,
wherein said wrapper is an elongated rectangular-shaped material which is sufficiently flexible to allow said wrapper to be wrapped around the circumference of said stent when said stent is stretched, and which length is at least as long as the length of the stent when the stent is stretched;
wherein said catheter is a tube shape with a proximal end and a distal end, said catheter defining a central opening there through from the proximal end to said distal end of said catheter, and wherein said stent in an elongated helical shape, with said wrapper wrapped around said stent from the proximal to the distal ends of said stent, can be placed in the central opening defined by said catheter; and
wherein when said stent is in an elongated helical shape and enclosed in said wrapper within said catheter, said stent may be pulled in or out of said catheter by pulling on an end of said wrapper.

3. A vascular assembly of claim 2 further comprising a first and second lock strips which lock strips are attached to the long edges of and a part of the wrapper and wherein said lock strips function to lock together when said stent and wrapper are within said catheter, and configured to release when said stent and said wrapper are not contained within said catheter.
